(19) United States
(12) Patent Application Publication
(10) Pub. No.: US 2021/0378818 A1
(43) Pub. Date: Dec. 9, 2021

(54) DEVICES AND METHODS FOR REPAIR OF VALVULAR INSUFFICIENCY

(71) Applicant: Edwards Lifesciences Corporation, Irvine, CA (US)

(72) Inventors: Boaz Manash, Givat Ada (IL); Noa Axelrod Manela, Caesarea (IL); David Linsky, Haifa (IL)

(21) Appl. No.: 17/412,211

(22) Filed: Aug. 25, 2021

Related U.S. Application Data
(63) Continuation of application No. PCT/US2020/018863, filed on Feb. 19, 2020.
(60) Provisional application No. 62/809,903, filed on Feb. 25, 2019.

(51) Int. Cl. A61F 2/24

(52) U.S. Cl. CPC A61F 2/246; A61F 2/2463; A61F 2220/0075; A61F 2220/005; A61F 2/2466

(57) ABSTRACT

Devices and methods for repairing a regurgitant heart valve are provided. A blocking component is fastened to a regurgitant valve such that the blocking component situates within the aperture of a regurgitant valve to mitigate the backflow across the valve. A variety of blocking components, fasteners, and delivery systems are described.

### RELATED APPLICATION

The present application is a continuation of PCT application PCT/US2020/018863 filed on Feb. 19, 2020, which claims the benefit of U.S. provisional patent application Ser. No. 62/809,903 filed on Feb. 25, 2019, titled DEVICES AND METHODS FOR REPAIR OF VALVULAR INSUFFICIENCY, each of which being incorporated herein by reference in its entirety.

### FIELD OF THE INVENTION

The application is generally directed to devices and methods to repair native valves, and more specifically to devices and methods that repair valvular insufficiency.

### BACKGROUND

Heart valves (such as the aortic, pulmonary, tricuspid and mitral valves) serve critical functions in assuring the forward flow of an adequate supply of blood through the cardiovascular system. These heart valves can be rendered less effective by congenital, inflammatory, or infectious conditions, resulting in regurgitation (i.e., backflow through the valve). Such conditions can eventually lead to serious cardiovascular compromise or death, and may require surgical repair and replacement of the valve.

Valvular insufficiency, such as aortic insufficiency (Al) (also referred to as aortic regurgitation), is a relatively common condition in which a native valve, such as the aortic valve, is unable to fully close, resulting in backflow leakage. For example, Aortic insufficiency can result in backflow of blood from the aorta into the left ventricle during diastole. Cardiac output is reduced due to backflow, often resulting in enlarged or weakened cardiac muscle from working harder to produce sufficient blood flow to the extremities.

### SUMMARY OF THE INVENTION

Many embodiments are directed to devices and methods to repair valvular insufficiency. Several embodiments are directed to devices that can situate within an aperture, opening, or gap of a regurgitant heart valve such that regurgitation is mitigated. Embodiments are also directed to methods, including surgical and other medical procedures that situate a blocking component within a regurgitant heart valve. The blocking components can be expandable and contractible, e.g., expanding to block regurgitant blood flow, but contracting or compressing to allow more blood flow in the proper direction. Any and all of the methods, techniques, steps, etc. described herein can be performed on a living animal or on a non-living cadaver, cadaver heart, simulator, anthropomorphic ghost, etc.

In some embodiments, a blocking component for the repair of valvular insufficiency includes a bulky blocking component body adapted to situate within an aperture, opening, or gap of a regurgitant heart valve, and configured such that when situated within the opening or gap, the bulky blocking component body fills the opening/gap (e.g., all or part of the opening/gap) and mitigates regurgitant flow. In one embodiment, the bulky blocking component body is made of a flexible and conformable material such that when situated within the opening/gap, the bulky blocking component body responds to the cycles of systole and diastole by expanding to fill the opening/gap when the local pressure at the valve is low, mitigating regurgitant flow, and contracting when the local pressure at the valve is high, allowing the forward flow of blood.

In some embodiments, a blocking component also includes a fastener such the fastener is adapted to anchor the blocking component within the opening/gap by securing the bulky blocking component body within a valve.

In one embodiment, the fastener is selected from the group consisting of: an adhesive, a set of one or more sutures, and a set of one or more clips.

In one embodiment, the fastener anchors the bulky blocking component body to a set of one or more native leaflets of the valve.

In various embodiments, the fastener is a clip that is secured onto the bulky blocking component body by a connective element selected from the group consisting of: sutures, adhesives, and staples. When the clip anchors the bulky blocking component body, the clip attaches (e.g., crimps, clips, etc.) onto a set of one or more native leaflets of the heart such that the bulky blocking component body anchors within the opening/gap of the valve.

In one embodiment, the clip includes an eye that can accommodate a wire therethrough such that the clip can be opened by tautening (or pulling taut) the wire.

In one embodiment, the bulky blocking component body and fastener are incorporated into a delivery system.

In some embodiments, the delivery system is a transcatheter delivery system further includes a catheter housing the bulky blocking component body and fastener within, and a guide wire that passes through and extends beyond the catheter and provides a means to guide the catheter through a patient's cardiovascular system.

In various embodiments, the fastener is a clip that is secured onto the bulky blocking component body by a connective element selected from the group consisting of: sutures, adhesives, and staples, such that the clip includes an eye that can accommodate a wire therethrough, and such that the transcatheter delivery system further comprises an actuating wire that passes through the eye and is adapted to open the clip.

In various embodiments, the bulky blocking component is formed from a material selected from a group consisting of: pericardium, expanded polytetrafluoroethylene (ePTFE), polyethylene terephthalate (PET), nylon, and polymer foam.

In some embodiments, a blocking component for the repair of valvular insufficiency includes an opening and closing blocking component body (e.g., a pocket-like blocking component body, pocketed blocking component body, pouch-like blocking component body, leaflet-like blocking component body, etc.) adapted to situate within an aperture, opening, or gap of a regurgitant heart valve. In some embodiments, the opening/closing blocking component body has a pocket or pouch (or other opening/closing flap) with upper perimeter dimensions such that when the blocking component body is situated within the opening/gap. The upper perimeter of the pocket can open and extend into and fill the opening/gap (e.g., all or part of the opening/gap) and mitigate regurgitant flow. The opening/closing blocking component body can be made of a flexible and conformable material such that when situated within the opening/gap, the blocking component body responds to the cycles of systole and diastole by opening and expanding to fill the opening/gap when the local pressure at the valve is low, mitigating regurgitant flow, and closing and contracting when the local pressure at the valve is high, allowing the forward flow of blood.

In some embodiments, a blocking component also includes a fastener such the fastener is adapted to anchor the blocking component body within the opening/gap by securing the blocking component body within a valve.

In various embodiments, the fastener is selected from the group consisting of: an adhesive, a set of one or more sutures, and a set of one or more clips.

In one embodiment, the fastener anchors the blocking component body to a set of one or more native leaflets of the valve.

In various embodiments, the fastener is a clip that is secured onto the blocking component body by a connective element selected from the group consisting of: sutures, adhesives, and staples. When the clip anchors the blocking component body, the clip attaches (e.g., crimps, clips, etc.) onto a set of one or more native leaflets of the heart such that the blocking component body anchors within the opening/gap of the valve.

In one embodiment, the clip includes an eye that can accommodate a wire therethrough such that the clip can be opened by tautening (or pulling taut) the wire.

In one embodiment, the blocking component body and fastener are incorporated into a delivery system.

In some embodiments, the delivery system is a transcatheter delivery system further including a catheter housing the blocking component body and fastener within and a guide wire that passes through and extends beyond the catheter and provides a means to guide the catheter through a patient's cardiovascular system.

In various embodiments, the fastener is a clip that is secured onto the blocking component body by a connective element selected from the group consisting of: sutures, adhesives, and staples such that the clip includes an eye that can accommodate a wire therethrough and such that the transcatheter delivery system further comprises an actuating wire that passes through the eye and is adapted to open the clip.

In various embodiments, the blocking component body is formed from a material selected from a group consisting of: pericardium, expanded polytetrafluoroethylene (ePTFE), polyethylene terephthalate (PET), nylon, and polymer foam.

In some embodiments, a method of mitigating valvular insufficiency includes situating a blocking component within an aperture, opening, or gap (e.g., a region of the valve that does not fully close) of a regurgitant heart valve such that the blocking component fills the aperture/opening/gap (e.g., all or part of the aperture/opening/gap) and mitigates regurgitant flow. The method can be performed on a living animal or on a non-living cadaver, cadaver heart, simulator, anthropomorphic ghost, etc. In some embodiments, the blocking component is made of a flexible and conformable material that responds to the cycles of systole and diastole by expanding to fill the opening/gap when the local pressure at the valve is low, mitigating regurgitant flow, and contracting when the local pressure at the valve is high, allowing the forward flow of blood. In one embodiment, the blocking component can include a bulky blocking component body and/or an opening/closing blocking component body. The bulky blocking component body is configured to fill the opening/gap and mitigate regurgitant flow. The opening/closing blocking component body can be a pocket or pouch-like blocking component body that has a pocket or pouch with upper perimeter dimensions configured to open extend into and fill the opening/gap (e.g., all or part of the opening/gap) and mitigate regurgitant flow.

In various embodiments, the method also includes anchoring the blocking component within the valve using a fastener such that the fastener is selected from the group consisting of: an adhesive, a set of one or more sutures, and a set of one or more clips.

In one embodiment, the fastener anchors the blocking component to a set of one or more native leaflets of the valve.

In various embodiments, the fastener is a clip that is secured onto the blocking component by a connective element selected from the group consisting of: sutures, adhesives, and staples. The method also includes attaching (e.g., crimping, clipping, etc.) the clip onto a set of one or more native leaflets of the valve such that the blocking component anchors within the aperture, opening, or gap of the valve.

In one embodiment, the clip includes an eye that can accommodate a wire therethrough. The method also includes opening the clip by tautening (or pulling taut) the wire.

In some embodiments, a method to repair valvular insufficiency includes approaching, via a patient's circulatory system, a regurgitant heart valve with a transcatheter delivery system. The method can be performed on a living animal or on a non-living cadaver, cadaver heart, simulator, anthropomorphic ghost, etc. The transcatheter delivery system includes a blocking component adapted to situate within an opening/gap within the regurgitant valve and configured such that, when situated within the opening/gap, the blocking component mitigates regurgitation across the valve. The transcatheter delivery system can also include a fastening clip secured to the blocking component. The fastening clip can have an eye. The transcatheter delivery system can also include an actuating wire disposed through the eye. In one embodiment, the transcatheter delivery system also includes a catheter defining an internal volume into which the blocking component, the fastening clip, and/or the actuating wire are disposed.

In some embodiments, the method to repair valvular insufficiency also includes advancing the blocking device and the clip with actuating wire out of the catheter and towards a native leaflet proximate to the opening/gap. The method can also include opening the clip by tautening (or pulling taut) the actuating wire and attaching (e.g., crimping, clipping, etc.) the clip to the native leaflet such that the blocking component is disposed in a configuration to fill the opening/gap (e.g., all or part of the opening/gap) and mitigate regurgitant flow.

In one embodiment, the method to repair valvular insufficiency also includes creating an incision in a blood vessel at a site distal from the valve to insert the transcatheter delivery system in order to approach the valve.

In various embodiments, the blood vessel is a femoral, a subclavian, or a carotid artery.

In one embodiment, the method to repair valvular insufficiency also includes viewing the blocking and device at the valve site using an imaging technique selected from: fluoroscopy and echocardiogram.

In one embodiment, the imaging technique is used to ensure that the clip is secured (e.g., crimped, clipped, etc.) on the native leaflet and that the blocking component is disposed in the configuration to fill the opening/gap and mitigate regurgitant flow.

Additional features and embodiments are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides an illustration of a human heart.

FIG. 2A provides an illustration of a healthy human heart with an aortic valve fully closing.

FIG. 2B provides an illustration of a human heart with a regurgitant aortic valve commiserate with aortic insufficiency.

FIG. 3A provides a detailed illustration of a regurgitant aortic valve.

FIG. 3B provides a detailed illustration of a regurgitant aortic valve with an example of a blocking component mitigating the regurgitation.

FIG. 4A provides a perspective view illustration of an example of an opening and closing pocket-like blocking component.

FIG. 4B provides a top view illustration of the example blocking component of FIG. 4A.

FIG. 4C provides a front view illustration of the example blocking component of FIG. 4A.

FIG. 5A provides a perspective view illustration of an example of a bulky gap-filler blocking component.

FIG. 5B provides a top view illustration of an example of a bulky gap-filler blocking component.

FIG. 5C provides a front view illustration of an example of a bulky gap-filler blocking component.

FIGS. 6A to 6C provide an illustration of an example of an opening/closing blocking component responding to cycles of systole and diastole.

FIGS. 7A to 7C provide an illustration of an example of a bulky gap-filler blocking component responding to cycles of systole and diastole.

FIGS. 8 to 14 provide illustrations of various example clips usable for attaching a blocking component to a valve.

FIGS. 15A to 16B provide illustrations of example clips adjoined to example blocking components.

FIGS. 17A and 17B provide illustrations of example delivery devices for delivering a blocking component to a valve.

FIGS. 18A to 18D provide detailed illustrations of an example of clip actuation using a wire.

FIGS. 19A to 19E provide illustrations of a medical procedure that can be performed in accordance with various embodiments.

### DETAILED DESCRIPTION

Turning now to the drawings, devices and methods to mitigate and/or prevent heart valve regurgitation are described, in accordance with various embodiments of the invention. In many embodiments, a regurgitation mitigation device that incorporates a blocking component and a fastener (e.g., a clip) is used to treat regurgitation by fastening the device to a heart valve to occupy gaps that exist in a leaky valve. Several heart valves can be repaired in accordance with methods and embodiments as described herein, including the aortic, tricuspid, mitral, and pulmonary valves.

A number of embodiments are directed towards design and function of blocking components. Accordingly, various embodiments of a blocking component can take various different conformations but are essentially to have the function of mitigating valve regurgitation. To mitigate valve regurgitation, blocking components provide a means to prevent backflow of fluid across a heart valve. In some embodiments, a blocking component comprises a leaflet-like pocket capable of expanding and contracting, like the native leaflets of heart valves, and thus provides a pocket to capture the backflow of fluids. Embodiments are also directed towards a blocking component that is bulky, yet flexible, and acts as a gap-filler that can conform to the opening/gap of a leaky valve in order to fill it.

A blocking component, in accordance with numerous embodiments, is made of a material that provides flexibility, durability and biocompatibility. In many embodiments, a blocking component is made of a material that enables it to conform to gaps existing in a leaky heart valve. Embodiments are also directed to blocking components made of materials that allow expansion and contraction of the blocking component, as to assist in form the open and closed states of a heart valve in accordance to the regular cycles of systole and diastole. A number of materials can provide these attributes, including (but not limited to) pericardium, expanded polytetrafluoroethylene (ePTFE), polyethylene terephthalate (PET), nylon, polymer foam, and other polymers having desirable properties.

Various embodiments are directed to the use of a fastener that functions to attach a blocking component to one or more valve leaflets or cusp(s) such that the blocking component can provide a means to prevent backflow regurgitation. Sutures, adhesives and/or clips can be used as a fastener in accordance with various embodiments. Clips can be provided in a number variable designs. In several embodiments, a clip is designed to robustly secure or attach (e.g., crimp, clip, etc.) to a valve leaflet or cusp such that the blocking component can maintain in its location within the heart valve for several years, decades, or even a lifetime without causing detrimental harm to the recipient. In a number of embodiments, a clip can be made of a material, which can provide these other desired attributes. Embodiments of clip materials include nitinol, cobalt-chrome (CoCr), stainless steel (e.g., 316L), titanium, various polymers, and other materials that provide desirable attributes.

Many embodiments utilize a transcatheter delivery system to deliver a blocking component to a regurgitant valve. Accordingly, a number of embodiments of a blocking component fit within a catheter such that the blocking component can be delivered to the heart valve to be repaired. Embodiments are also directed to a delivery device capable of actuating a clip such that the clip can open and then precisely crimp or clip onto a heart valve leaflet or cusp to locate a blocking component. Use of a transcatheter delivery device allows blocking components and clips to be delivered by minimally invasive procedures in which a small incision in a recipient at site distal to the heart, utilizing the circulatory system to reach the heart valves. In some embodiments, a transfemoral, subclavian, transapical, or transaortic approach is used. It should be noted, however, that an open-heart surgery can be used in some embodiments.

The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus. Additionally, any and all of the methods, operations, techniques, steps, etc. described herein can be performed on a living animal or on a non-living cadaver, cadaver heart, simulator, anthropomorphic ghost, etc.

### Overview of Leaky Heart Valves and Valvular Insufficiency

Embodiments of devices and methods are directed towards repair of leaky heart valves by mitigating regurgitation. Several of the figures and accompany descriptions relate to aortic insufficiency and devices and methods to repair a leaky aortic valve. It should be understood, however, that various embodiments that are provided to repair a leaky aortic valve can be used to treat other heart valves, such as the tricuspid, mitral, and pulmonary valves, as appropriate and understood by those having ordinary skill in the art. Accordingly, numerous embodiments should not be viewed to be limited to devices and methods to repair a leaky aortic valve, but should be expanded to repairs of other heart valves.

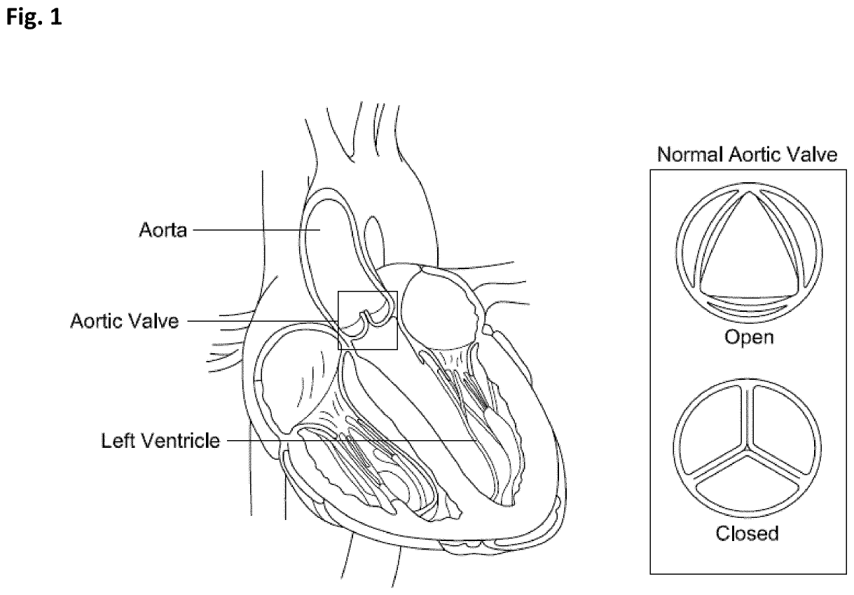

Depicted in FIG. 1 is a cross-sectional view of the left ventricle and aorta of a typical human heart. As can be seen, the aortic valve connects the left ventricle to the aorta. The aortic valve has an ability to open and close, responding to the pressures involved in systolic and diastolic rhythm, and ensuring directional flow of blood in the circulatory system.

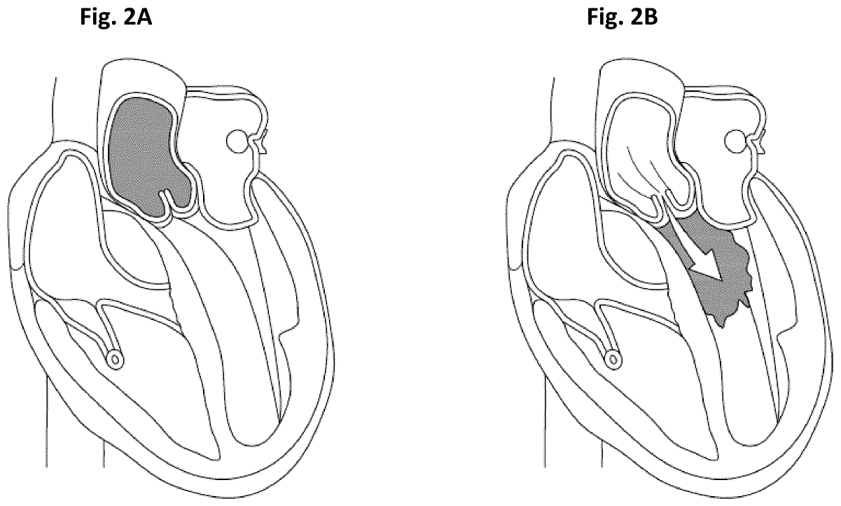

In a healthy functioning heart, the left ventricle contracts (i.e., systole) increasing the pressure within the ventricle causing the aortic valve to open and allowing blood to enter the aorta for whole body distribution. Directly after left ventricular contraction, the pressure in the ventricle drops (i.e., diastole), forcing the aortic valve to close and ensuring that the blood flows in an outward direction (see FIG. 2A). If the aortic valve fails to close, blood can regurgitate back into the left ventricle (see FIG. 2B).

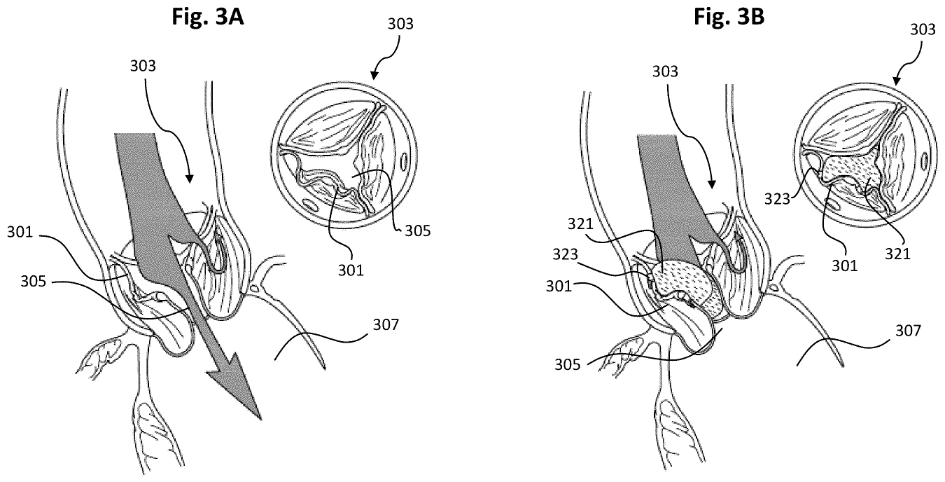

Aortic insufficiency (Al), also referred to as aortic regurgitation (AR), arises when one or more leaflets or cusps (301) of the aortic valve (303) is unable to fully close during diastole, leaving a regurgitant opening/gap (305) within the valve (FIG. 3A). The inability to close allows blood within the aorta to regurgitate back into the left ventricle (307), resulting in various complications including reduced cardiac output. Valvular insufficiency can occur in other heart valves as well, resulting in tricuspid, mitral, and pulmonary regurgitation, respectively.

Some attempts to treat valvular insufficiency involve surgical procedures to suture together leaflets or cusps of a valve, surgical procedures to replace the aortic root (e.g., the David Procedure), reconstructing the valve by replacing the valve with a prosthetic, or the use of a prosthetic ring stent to decrease the annulus of the valve. Each of these solutions do have their problems and may not be appropriate for all patients suffering from Al. For example, the David procedure is a very advanced and risky procedure that is more appropriate for younger patients with inherited disorders. Other complication arise with the use of prosthetic valve replacements and ring stents, as these may prevent further surgeries from being performed, if further complications surrounding the valve area arise. Accordingly, there is a need for an alternative approach to overcome these potential complications.

Several embodiments herein are directed to the use of a blocking component to mitigate valvular regurgitation by filling the gap that exists in the aortic valve of a patient having Al (and can be applied to other valves having insufficiency issues as well). As shown in an embodiment, in FIG. 3B, a blocking component (321) is inserted within a gap/aperture/opening (305) that is present within the aortic valve (303). In this embodiment, as is the goal with several embodiments as described herein, a blocking component (321) is designed to fill all the space of the opening/gap (305) to reduce and/or prevent regurgitation from occurring (see FIG. 3B). In many embodiments, a blocking component is made of a flexible and conformable material capable of filling the open space within the aortic valve. In some embodiments, a blocking component can fill all appreciable gaps within the aortic valve, preventing all regurgitation. Embodiments are also directed to a blocking component capable of reducing valvular regurgitation by 10%, 20%, 30%, 40%, 50%, 60%, 70%, 80%, 90%, 95%, or 99%.

To secure a blocking component within an aortic valve gap, multiple embodiments utilize a fastening mechanism. In the embodiment portrayed in FIG. 3B, clips (323) are attached to the blocking component (321) that are capable of crimping or clipping onto at least one of the aortic valve leaflets (301). It should be understood that a number of fastening mechanisms could be used to secure a blocking component, including (but not limited to) mechanical clips, spring clips, adhesives, sutures, and attachment to a prosthetic stent by an appropriate means.

While specific implementations of mitigating valve regurgitation using a blocking component are illustrated in FIG. 3B and described above, one of ordinary skill in the art can appreciate that various implementations can be used to mitigate valve regurgitation and that certain aspects may be optional according to some embodiments of the invention. As such, it should be clear that a number of implementations to mitigate valve regurgitation could be used as appropriate to the particular requirements of specific applications taking into consideration the medical procedure and needs of a patient. Furthermore, a variety of ways of mitigating valve regurgitation using a blocking component appropriate to the requirements of a given application can be utilized in accordance with various embodiments of the invention.

It should also be noted that a blocking component can be utilized in conjunction with other devices and methods to mitigate valve regurgitation. Accordingly, embodiments are directed towards a system to mitigate regurgitation that incorporates a blocking component and at least one other mechanism to mitigate valve regurgitation. A number of other devices and methods to mitigate regurgitation are known that could be appropriate to use in conjunction with a blocking component. For example, structures that fasten together valve leaflets that effectively reduce valve annulus diameter can be used in conjunction a blocking component. Fastening structures, such as rings, clips, adhesives, and sutures, are often used to fasten leaflets together near the outer diameter of the valve, however a regurgitant opening/gap may still exist. Thus, a blocking component can be located with any regurgitant opening/gap to further reduce valve regurgitation. Description of rings, clips and adhesives to reduce regurgitation are described in U.S. Pat. No. 9,622,863 and U.S. Provisional Application No. 62/575,252, which are each herein incorporated by reference in its entirety.

### Blocking Components

In accordance with several embodiments, a blocking component functions to mitigate regurgitation in a heart valve by blocking the backflow of blood. A blocking component, in many embodiments, primarily serves to impede backflow by taking up the aperture or opening gap space within a regurgitant valve. To achieve this purpose, a blocking component will have a structure and construction capable of taking form of available space within a regurgitant valve aperture/opening/gap. In addition, various embodiments of a blocking component will further include a fastening mechanism such that the blocking component is secured within a regurgitating heart valve.

In various embodiments, a blocking component will have a flexible and adaptable structure to accommodate the space within a regurgitant aperture/opening/gap of a heart valve. In several embodiments, a blocking component has a leaflet-like structure that is capable of mimicking at least some aspects of a native valve leaflet. In many embodiments, a blocking component is a bulky gap-filler structure that is capable of conforming within an opening/gap of a heart valve such that the opening/gap is filled.

Various embodiments of opening and closing blocking components and leaflet-like blocking components incorporate a pocket or pouch that is capable of catching blood regurgitation, which can be similar to how a native leaflet catches blood and blocks regurgitation. Many of the embodiments of leaflet-like pockets can expand and contract. leaflet-like pockets can open to expand into gap areas within a native valve and contract in areas where the gap is closed. In addition, various embodiments of leaflet-like pockets are responsive to the cycles of diastole and systole. Abilities to respond and contract are portrayed in FIGS. 6A to 6C and described in the accompanying text.

When implanted within a valve, embodiments of an opening/closing or leaflet-like blocking component should be oriented such that the opening of the pocket or pouch is superior (i.e., facing upward) relative to the valve. The width and length of an opening/closing blocking component will vary, often depending on the size of the regurgitant opening/gap. The outer perimeter of an opening/closing blocking component often will be of a length that is capable of filling the regurgitant opening/gap such that backflow is prevented (or at least mitigated) from leaking across the valve. The depth of a pocket or pouch can vary, and in some cases can depend on the needs of the patient. In some embodiments, the depth of a pocket or pouch is similar to the depth of a pocket area formed by a native leaflet.

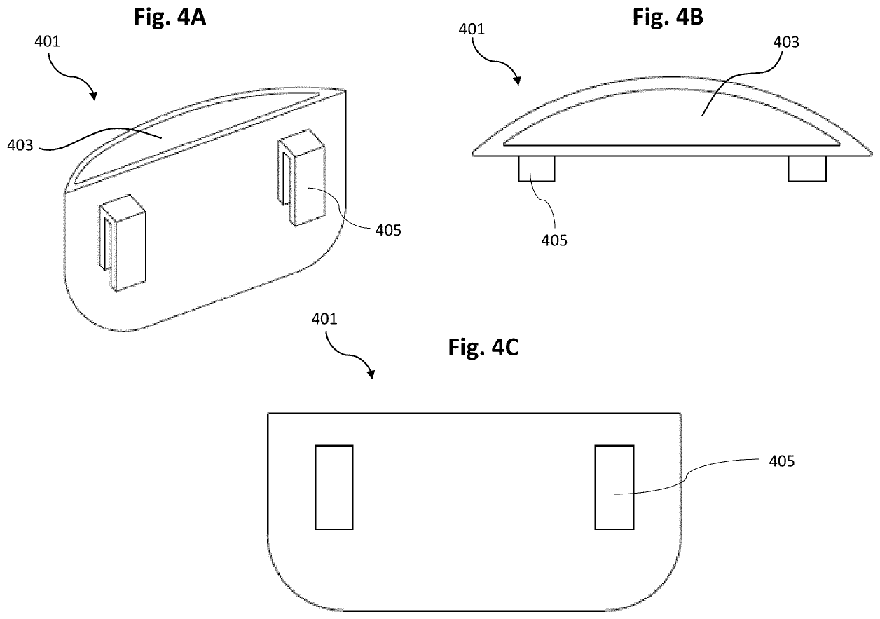

One embodiment of an opening/closing blocking component (401) that can be a leaflet-like blocking component is shown in perspective (FIG. 4A), plan (FIG. 4B) and elevation (FIG. 4C) views. The blocking component (401) has an openable/expandable and closable/contractible pocket or pouch (403), which can capture regurgitant backflow. Accordingly, when the device is implanted, the pocket (403) reacts to the pressures associated with the cycles of diastole and systole such that the pocket closes or contracts to the response of forward-flow pressure and opens or expands when the pressure is released to capture backflow and mitigate regurgitation.

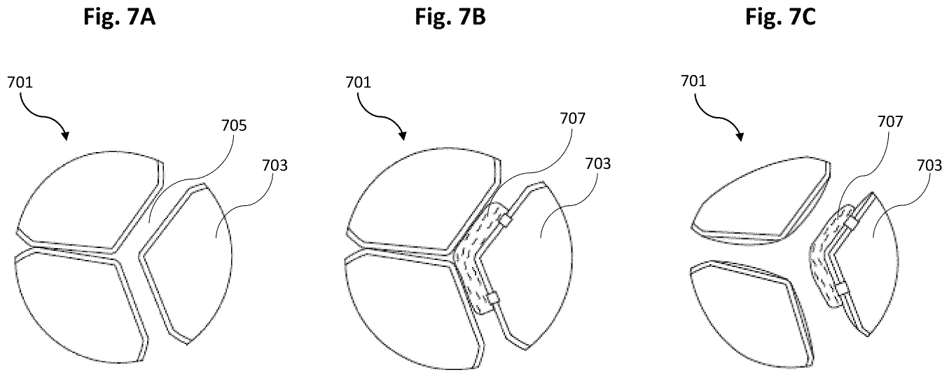

Various embodiments are directed a blocking component that utilizes a bulky gap filler to block regurgitant backflow of a regurgitant valve. In some embodiments, a bulky gap filler of blocking component has a cushion-like structure, capable of conforming to the shape of a regurgitant valve opening/gap. Furthermore, in a number of embodiments, a bulky blocking component responds to the pressures associated with the cycles of diastole and systole such that the gap filler contracts to the response of forward-flow pressure and expands when the pressure is released to block backflow and mitigate regurgitation, which is portrayed in FIGS. 7A to 7C.

In many embodiments, a bulky blocking component is incorporated within an opening/gap of a regurgitant valve and further is capable of expanding to fill the opening/gap such that backflow is impeded from leaking across the valve. The dimensions and size of a bulky blocking component can vary, and often depends on the size of the opening/gap to be repaired. Typically, a bulky blocking component will have a perimeter length that is capable of filling the regurgitant opening/gap such that backflow is prevented (or at least mitigated) from leaking across the valve.

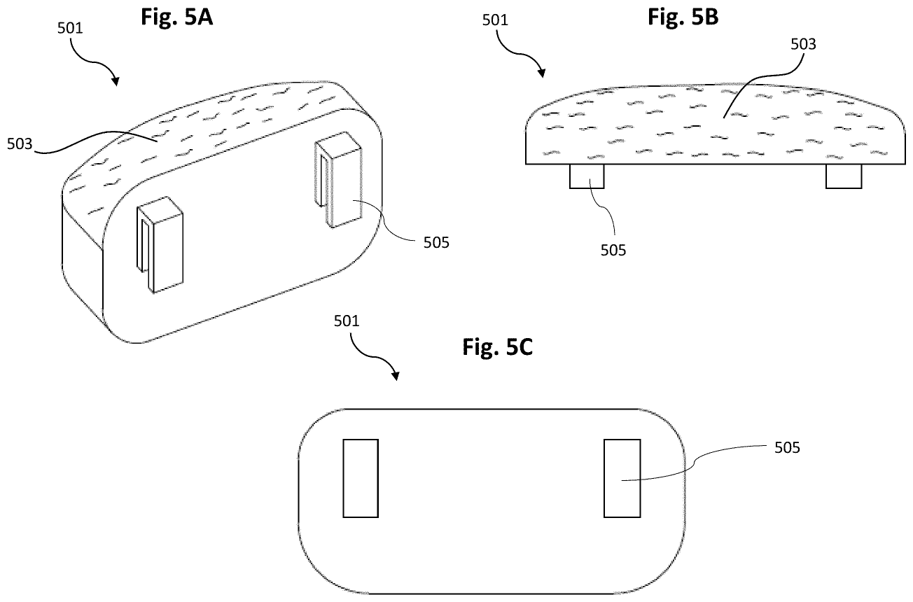

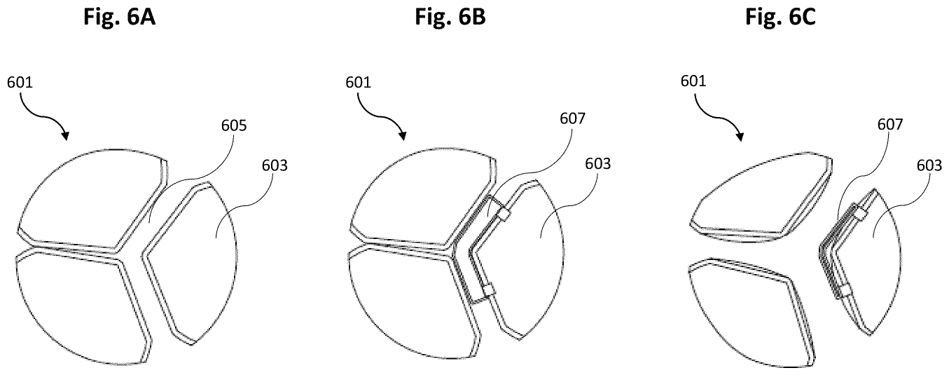

One embodiment of a bulky blocking component (501) is shown in perspective (FIG. 5A), plan (FIG. 5B) and elevation (FIG. 5C) views. The bulky blocking component has cushion-like shape (503), which can fill a regurgitant opening/gap to mitigate backflow. Accordingly, when the device is implanted, the bulky blocking component reacts to the cycles of diastole and systole such that the cushion-like shape contracts to the response of forward-flow pressure and expands when the pressure is released to block backflow and mitigate regurgitation.

Various embodiments of blocking components, including variations of opening/closing devices, leaflet-like devices, and bulky devices, are made of a biocompatible, flexible and durable material that is capable of conforming to the shape of the regurgitant opening/gap of a valve to be repaired. Ideally, these materials respond to changes of pressure associated with the cycles of diastole and systole such that a blocking component can expand when pressure is low and contract when pressure is high. Materials that can be used for blocking components include (but are not limited to) pericardium, expanded polytetrafluoroethylene (ePTFE), polyethylene terephthalate (PET), nylon, polymer foam, and other polymers having desirable properties.

A number of embodiments are also directed to mechanisms that fasten a blocking component within a regurgitant valve opening/gap. Various embodiments are directed to or involve the use of fastening mechanisms, such as clips, adhesives, sutures, clasps, etc., which are explained in greater detail in the corresponding section below. In several embodiments, a fastening mechanism is cooperative with a blocking component such that the blocking component can function as intended. Accordingly, fastening devices are adapted such that a blocking component can be situated within a valve opening/gap such that the blocking component can mitigate backflow across the valve.

In embodiments of blocking components that incorporate fastening mechanisms such as clips, a clip can be secured onto a blocking component by any appropriate mechanism. Appropriate mechanisms include (but are not limited to) sutures, staples, and adhesives. Provided in FIGS. 4A to 4C and 5A to 5C are examples of simple clips (405, 505) that are capable of clipping onto a native leaflet within the valve to be repaired. It should be noted, that although clips are depicted in FIGS. 4A to 4C and 5A to 5C, any appropriate fastening device can be incorporated in accordance with various embodiments.

A number of embodiments are directed to a blocking component that expands to fill a valve aperture, opening, or gap when pressure is low and contracts to allow forward flow when pressure is high in accordance with diastole and systole cycles. An example of this ability to contract and expand are provided in FIGS. 6A to 6C and 7A to 7C. Depicted in each of FIG. 6A and 7A is an insufficient valve (601, 701), presented in a top-down view. As can be seen in the figures, one of the three leaflets is unable to fully close (603, 703) resulting in an aperture, opening, or gap (605, 705) that would allow backflow regurgitation. To mitigate regurgitation, a blocking component can be inserted into the opening/gap (605, 705). In FIG. 6B, an embodiment of a leaflet-like opening/closing blocking component (607) is presented within the opening/gap (605). Likewise, an embodiment of a bulky blocking component (707) is presented within the opening/gap (705) in FIG. 7B. FIGS. 6B and 7B depict a valve when pressure is low (e.g., aortic valve during diastole) and thus the valve is in its closed state. When pressure is low, the blocking component (607, 707) is in its expanded state, occupying much of opening/gap (605, 705). By occupying much of the opening/gap, backflow regurgitation is mitigated. As forward pressure increases (e.g., aortic valve during systole), the valve (601, 701) opens up as the native leaflets compress (FIGS. 6C and 7C). At this time, the blocking component (607, 707) also compresses, allowing free forward flow of blood.

While specific implementations of blocking components are illustrated in FIGS. 4A to 7C and described above, one of ordinary skill in the art can appreciate that various designs of blocking components can be used to mitigate valve regurgitation and that certain aspects may be optional according to some embodiments of the invention. As such, it should be clear that a number of blocking components could be used as appropriate to the particular requirements of specific applications taking into consideration the medical procedure and needs of a patient. Furthermore, a variety of blocking components appropriate to the requirements of a given application, some which are not depicted, can be utilized in accordance with various embodiments of the invention.

### Fastening Mechanisms

A number of embodiments are directed to fastening a blocking component within the opening/gap of an insufficient heart valve. Various devices and mechanisms can be used to fasten a blocking component, including (but not limited to) the use of clips, sutures, adhesive, and any other mechanism appropriate to secure a blocking component within a valvular opening/gap.

A fastening mechanism, in accordance with several embodiments, is durable and biocompatible such that it can locate a blocking component within a valvular opening/gap for an extended period of time. In some embodiments, a blocking component is to be secured within an opening/gap permanently and thus a fastening mechanism should last a lifetime. In more embodiments, a blocking component is removable, but is to be secured within an opening/gap for an extended period of years. In some instances, the fastening mechanism is durable enough to last at least: 1, 2, 5, 10, 15, 20, 25, 30, 35, or 40 years. The length of time a fastening mechanism is to secure a blocking component will often depend on the needs of the patient receiving the implant.

In numerous embodiments, a blocking component is secured within a valvular opening/gap with sutures and/or adhesives. Various embodiments of blocking component are sutured and/or adhered onto one or more native leaflets such that the blocking component is situated within an opening/gap. In some instances, if a prosthetic is situated within the valvular area, a blocking component can be sutured or adhered to the prosthetic. Suturing and/or adhering to a prosthetic may be useful in instances when a prosthetic is used to reduce the effective diameter of an annulus, such as various prosthetic rings and clips as described in U.S. Pat. No. 9,622,863 (cited supra). A number of adhesives could be used, such as those described in U.S. Provisional Patent Application No. 62/575,252 (cited supra).

In many embodiments, one or more clips are used to secure a blocking component within a valvular opening/gap. In several of these embodiments, a clip is attached to a blocking component by any appropriate means. For example, a clip can be attached by sutures, adhesives, staples, and various combinations thereof. With a clip attached to a blocking component, a blocking component can be situated within a valvular opening/gap by crimping or clipping the clip onto one or more native leaflets or a prosthetic within the valve to be repaired.

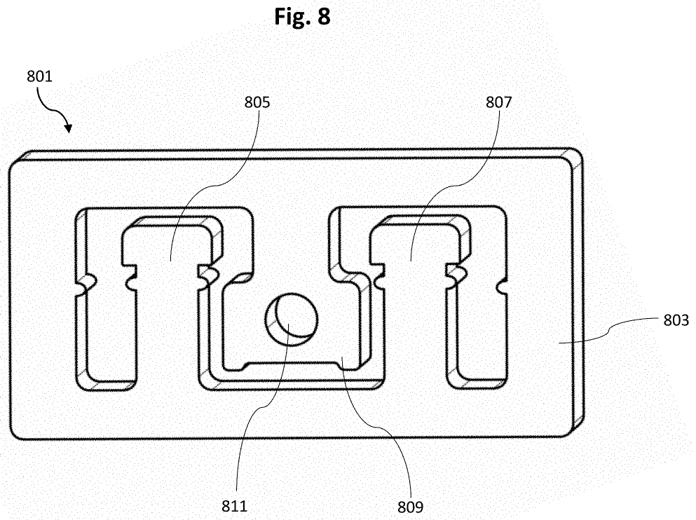

A variety of clip styles and designs may be used in accordance with various embodiments. A few specific embodiments are depicted in FIGS. 8A to 14D. Within these depicted embodiments are a number of features that may provide benefit (either directly or indirectly) in securing a blocking component within an opening/gap. It should be understood that although some of the depicted clips have a particular set of features, the various features depicted are not limited to particular clip design. Accordingly, various embodiments of clips are similar to those depicted, but may have some features may be removed, added, or exchanged.

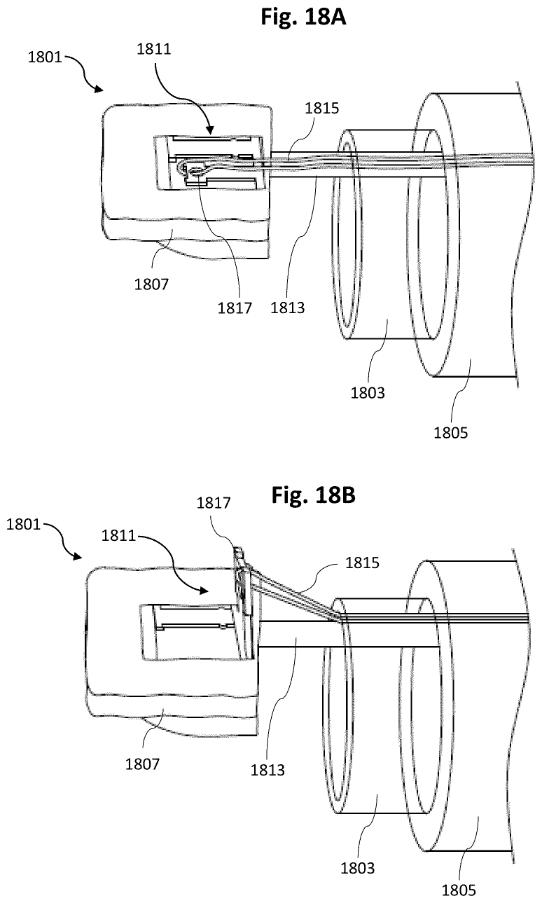

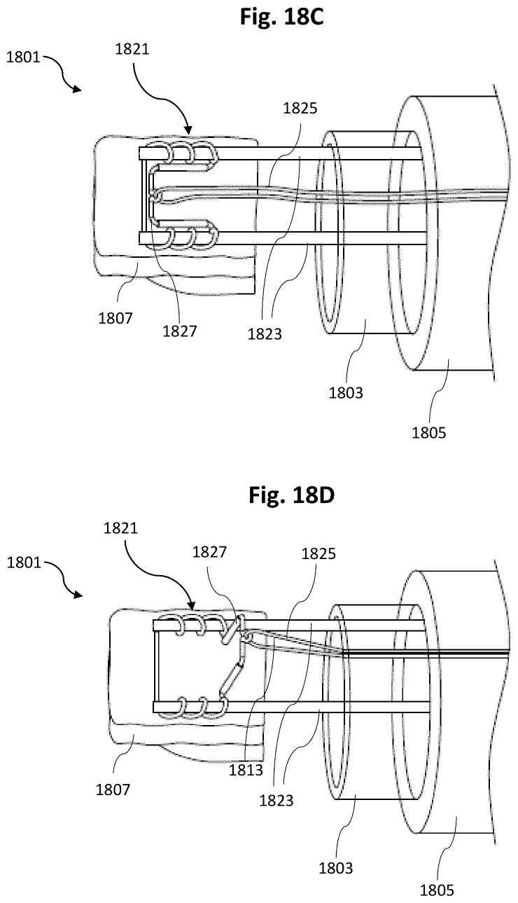

In several embodiments, a clip has an actuation mechanism such that a clip can be delivered to the site of repair in an open state (or opened at the site of repair), and then can be secured (e.g., crimped, clipped, etc.) onto an appropriate location. In many of these embodiments, a clip has an elastic force such that the elastic force keeps the clip in a closed and crimped or clipped position. Several structures are known to provide an elastic force, including (but not limited to) springs and wires. Furthermore, clips can be made of stiff materials (e.g., nitinol, CoCr, various polymers, and other metals) that have desirable elastic properties. To open clips that rely on elastic forces, several clip embodiments incorporate an eye or loop that a wire can be threaded or hooked in so that pulling on the wire opens the clip. Examples of actuating a clip within a delivery device utilizing at least some of these mechanistic concepts are depicted in FIGS. 18A to 18D and further detailed in the accompanying description.

An embodiment of a clip (801) is provided in perspective view (FIG. 8). In this embodiment, the clip (801) is a cutout of sheet metal to yield a frame (803), two outer prongs (805 and 807), and inner prong (809) having an eye (811). The frame (803) and two outer prongs (805) and (807) provide a structure to secure the clip to a blocking component by an appropriate means, such as sutures, adhesives, and/or staples. The inner prong (809) provides an elastic force such that in the closed flat position the clip (801) can crimp or clip onto a proper location, such as a native leaflet or prosthetic, to secure or attach the device. The eye (811) provides an actuation means to open the clip (801) by lifting the inner prong (809) in a direction normal to the plane of the clip face. To lift the inner prong (809) a wire or similar (not shown) is threaded or hooked through the eye (811) and the wire can be pulled taut to lift the inner prong (809). Release of the tension of the taut wire and/or unthreading the wire allows the clip (801) to crimp or clip and close. As shown in this embodiment, the inner prong (809) is proximate the outer prongs (805 and 807), which can help strengthen the crimping or clipping force.

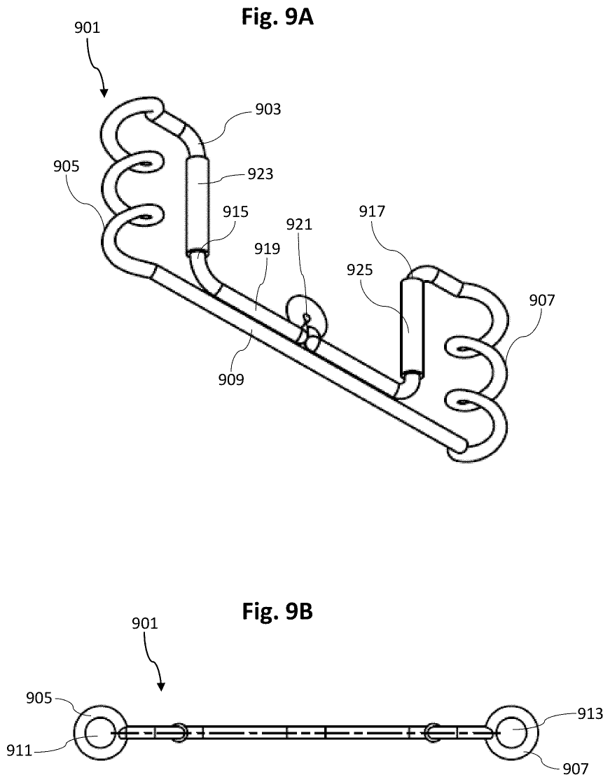

An embodiment of a clip (901) is provided in perspective (FIG. 9A) and top (FIG. 9B) views. In this embodiment, the clip (901) is formed into a wire frame (903). The clip wire has two side spirals (905 and 907) and an outer lower bar (909) that can assist in securing the clip (901) to a blocking component by allowing for a location to suture, adhere, and or staple the clip to a blocking component. The side spirals (905 and 907) also provide slots (911 and 913) within spiral that can be used to attach the clip to a delivery system. In one scenario, the delivery system utilizes two beams that can insert within the slots (911 and 913) to hold and secure the clip as it is delivered to the site of repair. The side spirals (905 and 907) can also provide grip to help anchor the clip when it is crimped or clipped onto a proper location. The clip has two inner side bars (915 and 917) and inner lower bar (919) that has and eye (921). Connecting elements (923 and 925), are situated on the two inner side bars (915 and 917), which can provide stability when crimping or clipping, segments for welding, radiopaque markers for viewing the clip during imaging, or any combination thereof. The connecting elements (923, and 925) can also be structural elements that stiffen the clip to enhance clip actuation, providing a virtual hinge when the clip opens and closes. The eye (921) provides an actuation means to open the clip (901) by lifting the inner lower bar (919) in a direction normal to the plane of the clip face. A wire or similar (not shown) is threaded or hooked through the eye (921) and the wire can be pulled taut to lift the inner lower bar (919). Release of the tension of the taut wire and/or unthreading the wire allows the clip (901) to crimp and close. As shown in this embodiment, the inner lower bar (919) is proximate the outer lower bar (909), which can help strengthen the crimping or clipping force.

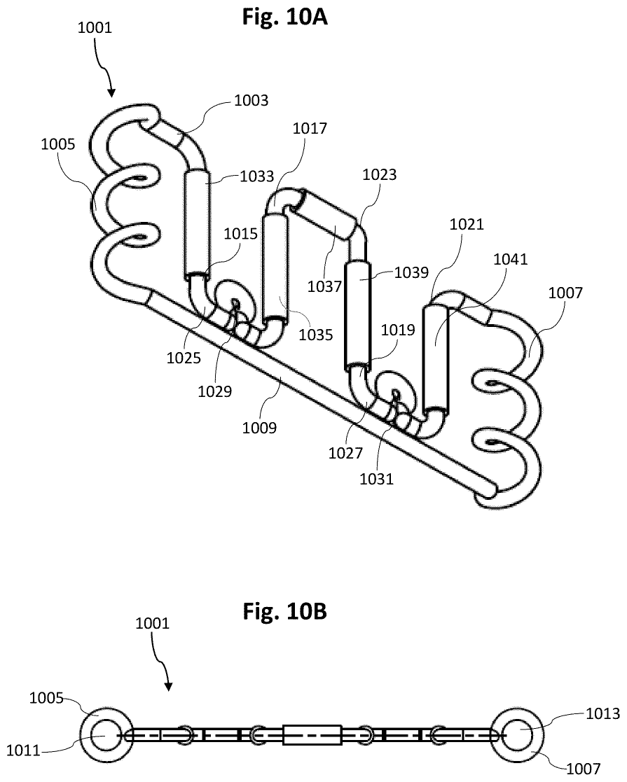

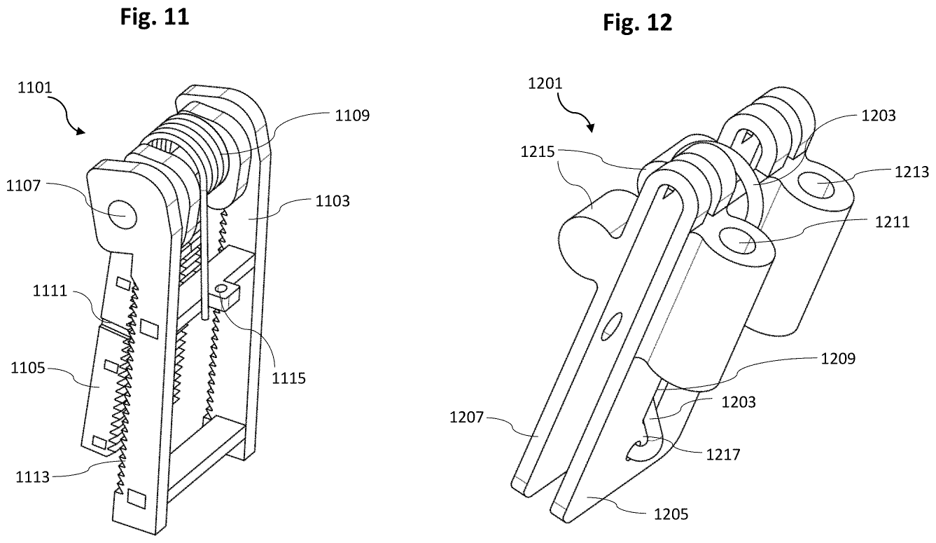

An embodiment of a clip (1001) is provided in perspective (FIG. 10A) and top (FIG. 10B) views. In this embodiment, the clip (1001) is formed into a wire frame (1003). The clip wire has two side spirals (1005 and 1007) and an outer lower bar (1009) that can assist in securing the clip (1001) to a blocking component by allowing for a location to suture, adhere, and or staple the clip to a blocking component. The side spirals (1005 and 1007) also provide slots (1011 and 1013) within spiral that can be used to attach the clip to a delivery system. In one scenario, the delivery system utilizes two beams that can insert within the slots (1011 and 1013) to hold and secure the clip as it is delivered to the site of repair. The side spirals (1005 and 1007) can also provide grip to help anchor the clip when it is crimped or clipped onto a proper location. The clip has four inner side bars (1015, 1017, 1019 and 1021), and inner upper bar (1023) and two inner lower bars (1025 and 1027) that each has and eye (1029 and 1031). Connecting elements (1033, 1035, 1037, 1039, and 1041), are situated on the four inner side bars (1015, 1017, 1019 and 1021) and the inner lower bar (1023), which can provide stability when crimping, segments for welding, radiopaque markers for viewing the clip during imaging, or any combination thereof. The connecting elements (1033, 1035, 1037, 1039, and 1041) can also be structural elements that stiffen the clip to enhance clip actuation, providing a virtual hinge when the clip opens and closes. The eyes (1029 and 1031) provide an actuation means to open both the left and right side the clip (1001), respectively, by lifting the inner lower bars (1025 and 1027) in a direction normal to the plane of the clip face. A wire or similar (not shown) is threaded or hooked through each of the eyes (1029 and 1031) and each wire can be pulled taut to lift the each inner lower bar (1025 and 1027) such that each side the clip (1001) can be opened independently of the other side. Release of the tension of one of the taut wires and/or unthreading one of the wires allows one side of the clip (1001) to crimp or clip and close. As shown in this embodiment, the inner lower bars (1025 and 1027) are proximate the outer lower bar (1009), which can help strengthen the crimping or clipping force. Having two independent actuation mechanisms on each side can improve the ability of a longer clip to crimp or clip onto its intended sites. Furthermore, a single clip of this design can crimp or clip onto two independent locations, such as two native leaflets of a valve.

An embodiment of a clip (1101) is provided in a perspective view (FIG. 11). In this embodiment, the clip (1101) is assembled from various parts to form front frame (1103) and rear frame (1105) that are connected using a bar (1107) inserted into sockets within the top portion of the front and rear frames. A spring (1109) also surrounds the connecting bar (1107) and the spring has a terminal portion extending to the outer face of the front frame (1103), which provides the elastic force to close the clip (1101). The rear frame (1105) has slots (1111) that can assist in suturing the clip (1101) to a blocking component and thus securing the clip to the blocking component. The inner face of both the front and rear frames (1103 and 1105) have teeth (1113) to assist in crimping or clipping and anchoring the clip to intending site. Also provided is an eye (1115) on the outer face of the front frame (1103). The eye (1115) provides an actuation means to open the clip (1101) by lifting the front frame (1103) in a direction normal to the plane of the front frame outer face. A wire or similar (not shown) is threaded or hooked through the eye (1115) and the wire can be pulled taut to lift the front frame (1103). Release of the tension of the taut wire and/or unthreading the wire allows the clip (1101) to crimp and close. As shown in this embodiment, the front frame (1103) is proximate the rear frame (1105), which can help strengthen the crimping or clipping force.

An embodiment of a clip (1201) is provided in a perspective view (FIG. 12). In this embodiment, the clip (1201) is formed from a unitary piece with a wire (1203). The wire (1203) travels along the outer faces of the front (1205) and rear (1207) plates and over the top of the clip, interlocked at a location (1209) to provide elastic force to close the clip (1201). The clip (1201) also has two built-in guide slots that can be used to attach the clip to a delivery system. In one scenario, the delivery system utilizes two beams that can insert within the slots (1211 and 1213) to hold and secure the clip as it is delivered to the site of repair. Two rounded protrusions (1215) provide ends to attach ends of a spring to the clip (1201). Also provided is a loop (1217) within the wire (1203) between the interlocked location (1209) and the end attachment point of the wire. The loop (1217) in the wire (1203) provides an actuation means to open the clip (1201) by lifting the front plate (1205) in a direction normal to the plane of the front outer face. A wire or similar (not shown) is threaded or hooked through the loop (1217) and the wire can be pulled taut to lift the front face (1205). Release of the tension of the taut wire and/or unthreading the wire allows the clip (1201) to crimp or clip and close. As shown in this embodiment, the front plate (1205) is proximate the rear plate (1207), which can help strengthen the crimping or clipping force.

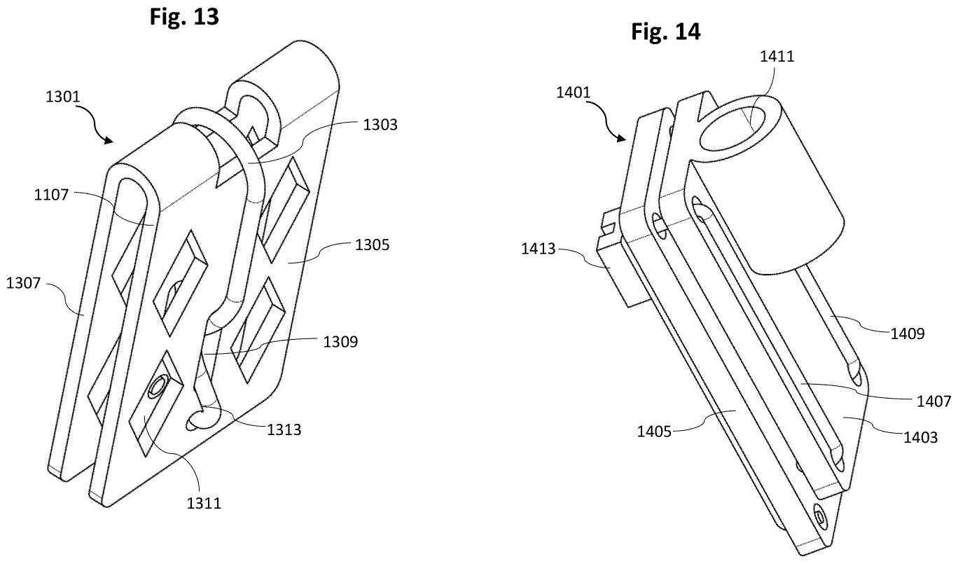

An embodiment of a clip (1301) is provided in a perspective view (FIG. 13). In this embodiment, the clip (1301) is formed from a unitary piece with a wire (1303). The wire (1303) travels along the outer faces of the front (1305) and rear (1307) plates and over the top of the clip, interlocked at a location (1309) to provide elastic force to close the clip (1301). The clip also has cut out portions (1311) on the front (1305) and rear (1307) plates which can be used to help suture or adhere a blocking component to the rear plate. Also provided is a loop (1313) within the wire (1303) between the interlocked location (1309) and the end attachment point of the wire. The loop (1313) in the wire (1303) provides an actuation means to open the clip (1301) by lifting the front plate (1305) in a direction normal to the plane of the front outer face. A wire or similar (not shown) is threaded or hooked through the loop (1313) and the wire can be pulled taut to lift the front face (1305). Release of the tension of the taut wire and/or unthreading the wire allows the clip (1301) to crimp or clip and close. As shown in this embodiment, the front plate (1305) is proximate the rear plate (1307), which can help strengthen the crimping force.

An embodiment of a clip (1401) is provided in a perspective view (FIG. 14). In this embodiment, the clip (1401) is formed from two pieces, one piece forming a front plate (1403) and the second piece forming a rear plate (1405), and two wires (1407 and 1409). The wires (1407 and 1409) travel along the outer faces of the front (1403) and rear (1405) plates and over the top of the clip to provide elastic force to close the clip (1401). The clip (1401) also has a guide slot (1411) that can be used to attach the clip to a delivery system. In one scenario, the delivery system utilizes a beam that can insert within the slot (1411) to hold and secure the clip as it is delivered to the site of repair. The clip can be actuated with a pull wire connected to back of rear plate (1405). As shown in this embodiment, the front plate (1403) is proximate the rear plate (1405), which can help strengthen the crimping or clipping force.

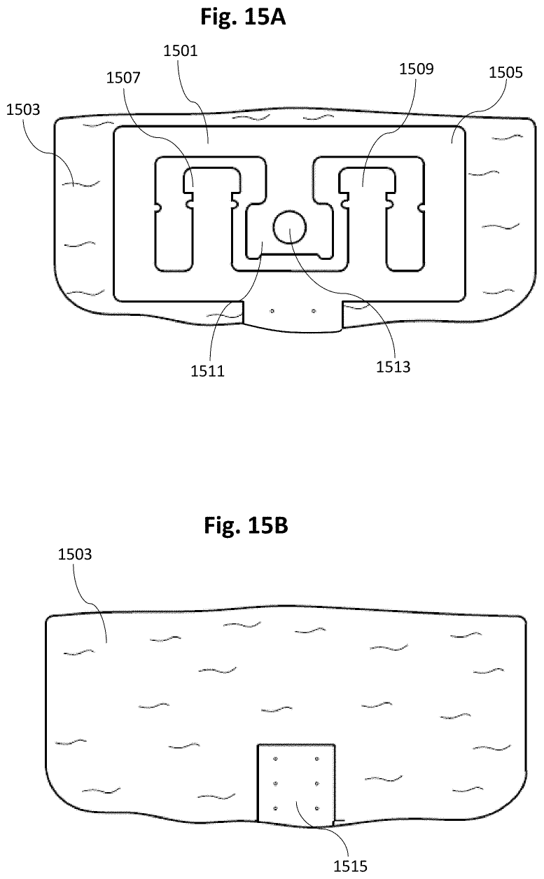

An embodiment of a clip (1501) attached to a blocking component (1503) is provided in front (FIG. 15A) and rear (FIG. 15B) views. The clip (1501), which is cut out of sheet metal, has a frame (1505), two outer prongs (1507 and 1509), and inner prong (1511) having an eye (1513). The frame (803) and two outer prongs (1507 and 1509) are secured to the blocking component (1503) by any appropriate means, such as sutures, adhesives, and/or staples. The inner prong (1511) is not attached to the blocking component (1503) so that it can provide a crimping or clipping function using an elastic force to crimp or clip the clip (1501) onto a proper location and thus situating the blocking component (1503) accordingly. On the rear side of the blocking component (1503), a pocket (1515) is attached by an appropriate means, such as sutures, adhesives, and/or staples. The pocket (1515) provides a slot that a beam from a delivery device can insert into, so that the blocking component (1503) can be held in place and secured as it is delivered to the site of repair.

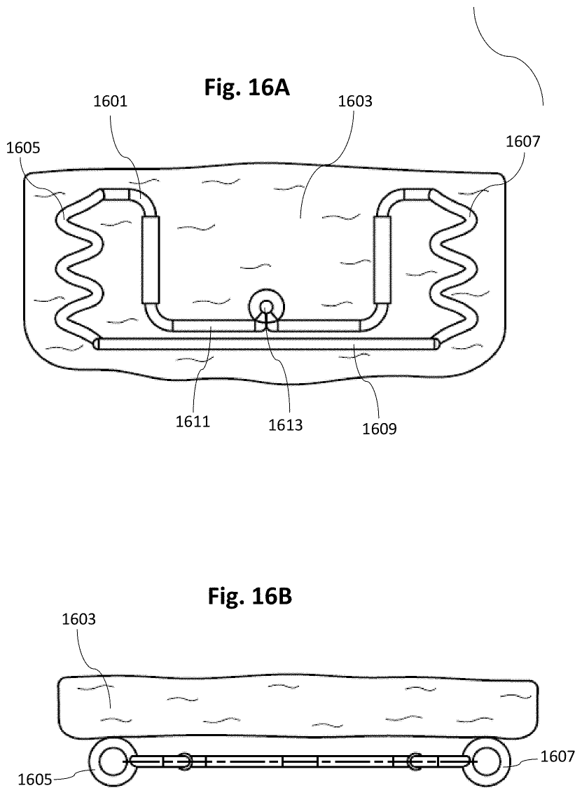

An embodiment of a clip (1601) attached to a blocking component (1603) is provided front (FIG. 16A) and top (FIG. 16B) views. The clip (1601) has a wire form with two side spirals (1605 and 1607) and an outer lower bar (1609) that are secured to the blocking component (1603) using sutures, adhesives, and or staples. The inner lower bar (1611) with an eye (1613) is not attached to the blocking component (1603) so that it can provide a crimping or clipping function using elastic force to crimp or clip the clip (1601) onto a proper location and thus situating the blocking component (1603) accordingly.

While specific implementations of clips are illustrated in FIGS. 8A to 16B and described above, one of ordinary skill in the art can appreciate that various designs of clips can be used to locate a blocking component and that certain aspects may be optional according to some embodiments of the invention. As such, it should be clear that a number of clips could be used as appropriate to the particular requirements of specific applications taking into consideration the medical procedure and needs of a patient. Furthermore, a variety of clips appropriate to the requirements of a given application, some which are not depicted, can be utilized in accordance with various embodiments of the invention.

### Delivery Systems and Implantation of Blocking Components

Systems to deliver and implant blocking components are provided in accordance with a number of embodiments of the invention. Generally, a number of embodiments utilize a delivery system in conjunction with a medical method to reach a regurgitant heart valve such that a blocking component can be implanted within the valve to mitigate the regurgitation. In many embodiments, minimally invasive surgery using a transcatheter delivery device is performed to deliver a blocking component via an artery or vein. A number of embodiments of transcatheter delivery devices are described in U.S. Pat. No. 9,622,863 (cited supra), many of which can be used and incorporated with various blocking component and clip embodiments described herein

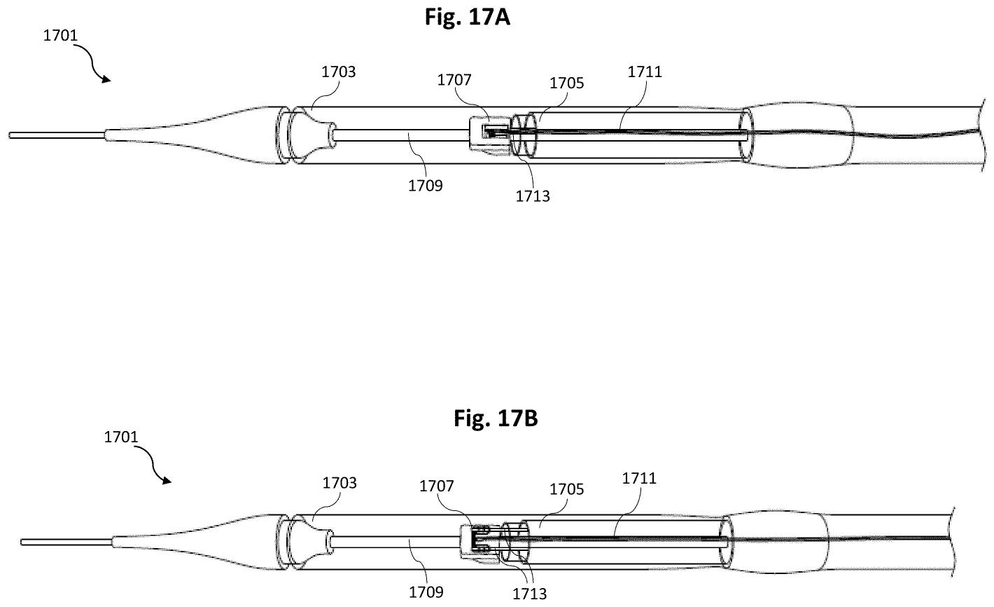

Provided in FIG. 17A and 17B are embodiments of a delivery device (1701) incorporating a blocking component to be delivered to the site of repair. In FIG. 17A, a blocking component with a clip cut from sheet metal is depicted. In FIG. 17B, a blocking component with a wire clip is depicted.

The delivery device (1701) has a flexible outer catheter (1703) and a flexible inner catheter (1705). The inner catheter houses the blocking component and clip (1707). Also provided is a flexible guide wire (1709) to transport the delivery device through the circulatory system. Within the inner catheter (1705) and looped through the eye of the clip is an actuating wire (1711), which is used to open the clip such that the clip can be crimped or clipped onto a leaflet or prosthetic at the site of repair. Also within the inner catheter (1705) is the support beam (1713) that secures the blocking component and clip (1707) as it is delivered.

FIGS. 18A to 18D detail the delivery device (1801) with the blocking component and clip advanced from the inner (1803) and outer (1805) catheters. In FIGS. 18A and 18B, a blocking component (1807) with a clip (1811) cut from sheet metal is depicted. In FIGS. 18C and 18D, a blocking component (1807) with a wire clip (1821) is depicted.

As can be seen in FIGS. 18A and 18B, a support beam (1813) provides support to the blocking component (1807) and clip (1811). When an actuating wire (1815) has slack, as depicted in FIG. 18A, the inner prong (1817) is down such that the clip is in a closed position. When the actuating wire (1815) is taught, as depicted in FIG. 18B, the inner prong (1817) is lifted upward such that clip is in an open position.

As can be seen in FIGS. 18C and 18D, two support beams (1823) provide support to the blocking component (1807) and clip (1821). When an actuating wire (1825) has slack, as depicted in FIG. 18C, the inner lower bar (1827) is down such that the clip is in a closed position. When the actuating wire (1825) is taught, as depicted in FIG. 18D, the inner lower bar (1827) is lifted upward such that clip is in an open position.

While specific implementations of delivery devices are illustrated in FIGS. 17A to 18D and described above, one of ordinary skill in the art can appreciate that various designs of delivery devices can be used to deliver a blocking component and that certain aspects may be optional according to some embodiments of the invention. As such, it should be clear that a number of delivery devices could be used as appropriate to the particular requirements of specific applications taking into consideration the medical procedure and needs of a patient. Furthermore, a variety of delivery devices appropriate to the requirements of a given application, some which are not depicted, can be utilized in accordance with various embodiments of the invention.

### Medical Procedures to Implant Blocking Components

Methods and procedures to implant blocking components are provided in accordance with a number of embodiments of the invention. Generally, embodiments of methods utilize a medical method to reach a regurgitant heart valve to implant a blocking component within the valve to mitigate the regurgitation. In some embodiments, a catheter is used to transvascularly navigate and deliver a blocking component to the valve via an artery or vein. In other embodiments, minimally invasive surgery via small insertions within the chest are performed to deliver a blocking component to the valve. Various embodiments employ open heart surgery to deliver a blocking component to the valve. And in some embodiments, a combination of medical procedures are performed. The precise medical method of delivery will vary and often depends on the procedure to be performed, the patient's condition, and the medical professional performing the procedure.

Several embodiments are directed towards a transvascular and/or transcatheter method to implant a blocking component at the site of regurgitant valve. Generally, transvascular and transcatheter procedures involve performing a small incision and inserting a catheter delivery system at site that is often distal from the heart, and transporting the delivery system to the heart via the circulatory system. In many embodiments, a transfemoral approach is used such that a small incision occurs in the femoral artery or femoral vein located in the groin or thigh. Various embodiments are also directed to transvenous, subclavian, transapical, transseptal, transatrial, transcaval, transaortic, and transradial approaches. In some embodiments, various valves can be reached via the subclavian and/or carotid arteries. It should be understood that any approach to reach repair a regurgitant valve can be used in accordance with various embodiments. To visualize the approach and repair, a number of methods can be used in accordance with various embodiments, including the use of fluoroscopy and echocardiogram imaging. For further description of transcatheter methods involving steps that can be used, refer to U.S. Pat. No. 9,622,863 (cited supra) and U.S. Pat. No. 6,908,481, which is herein incorporated by reference in its entirety.

Utilizing a medical method to deliver a blocking component to the site of repair, in accordance with numerous embodiments, the blocking component is to be localized within the regurgitant valve, and specifically within an opening/gap that is present when the valve is closed. The blocking component can then be implanted within the opening/gap to fill the vacated space. In several embodiments, a blocking component is held within the opening/gap by a fastening mechanism. Various embodiments of fastening mechanisms include (but are not limited to) the use of clips, adhesives, and sutures.

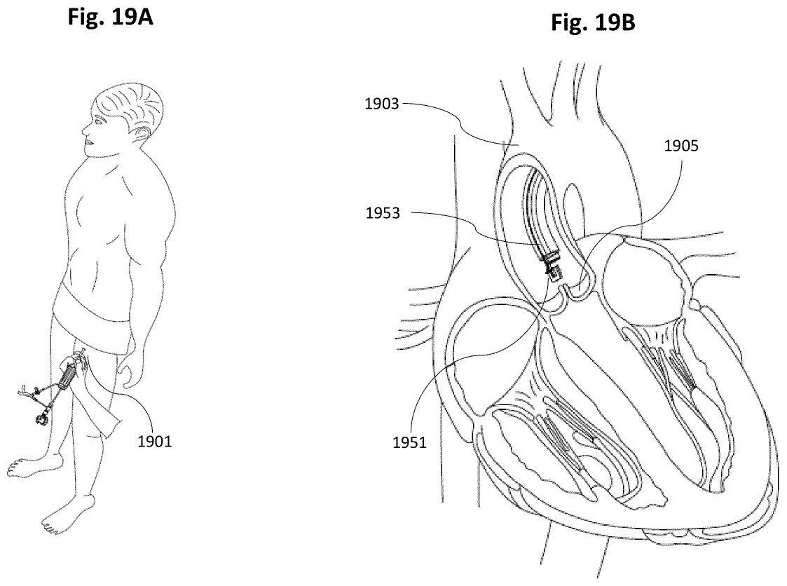

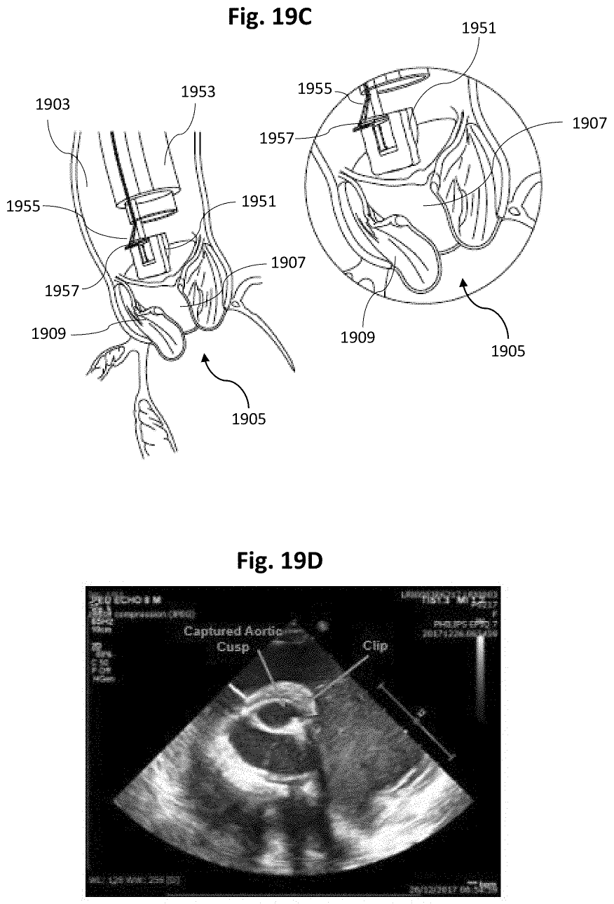

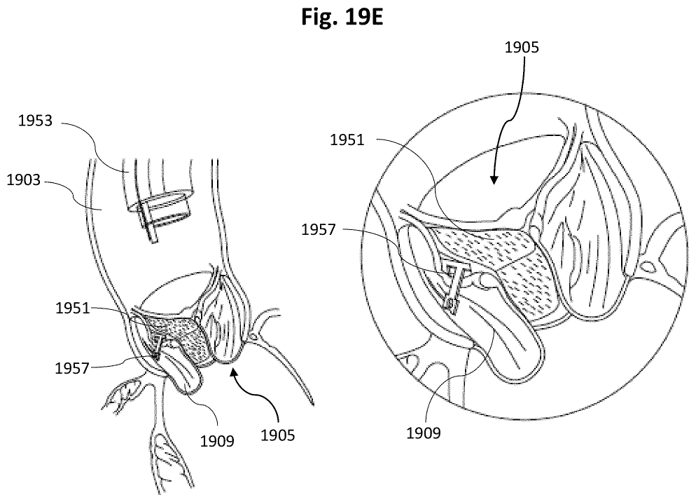

Provided in FIGS. 19A to 19E is an illustrated depiction of an embodiment of a transvascular and transcatheter method using a transfemoral (or similar approach) approach to reach the aortic valve. An incision is made in the groin area to reach the femoral artery (see FIG. 19A). A delivery device containing a blocking component is inserted at the incision site (1901). A guide wire is used to help navigate the delivery device through the arterial system and into aortic arch (1903). Once within the aortic arch or otherwise proximate the aortic valve, the blocking component (1951) is advanced out of the delivery device (1953) and towards the aortic valve (1905) and/or one or more of the leaflets thereof. Using the actuating wire (1955), the clip (1957) is opened as it approaches the valvular aperture/opening/gap (1907). In this embodiment, the clip (1957) is attached (e.g., crimped, clipped, etc.) onto a native leaflet (1909) by releasing the tension of the actuating wire. The actuation of the clip and implantation of the blocking component via attaching (e.g., crimping, clipping, etc.) of the clip onto a native leaflet can be visualized using an echocardiogram (see FIG. 19D). Once the clip (1957) is attached or clipped onto the native leaflet (1909) and the blocking component (1951) is situated within the opening/gap (see FIG. 19E), the delivery device (1953) recedes from the aortic valve and back out through the site of incision.

While a specific implementation of a transfemoral approach is illustrated in FIGS. 19A to 19E and described above, one of ordinary skill in the art can appreciate that various other approaches and procedures can be used to implant a blocking component at the aortic valve position or at other valve positions (e.g., tricuspid, pulmonary, and/or mitral valve positions) and that certain aspects may be optional according to some embodiments of the invention. As such, it should be clear that a number of approaches and procedures could be used as appropriate to the particular requirements of specific applications taking into consideration the devices to be implanted and the needs of a patient. Furthermore, a variety of steps of approaches and procedures appropriate to the requirements of a given application, some which are not depicted, can be utilized in accordance with various embodiments of the invention. Additionally, a transcatheter or other surgical approach can be performed on a living animal or on a non-living cadaver, cadaver heart, simulator, anthropomorphic ghost, etc.

### Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A blocking component for the repair of valvular insufficiency, comprising:
a bulky blocking component body adapted to situate within a gap of a regurgitant heart valve, and configured such that when situated within the aperture, the bulky blocking component body fills the aperture and mitigates regurgitant flow; and
wherein the bulky blocking component body is made of a flexible and conformable material such that when situated within the aperture, the bulky blocking component body responds to the cycles of systole and diastole by expanding to fill the aperture when the local pressure at the valve is low, mitigating regurgitant flow, and contracting when the local pressure at the valve is high, allowing the forward flow of blood.

2. The blocking component according to claim 1 further comprising a fastener;
wherein, the fastener is adapted to anchor the blocking component within the aperture by securing the bulky blocking component body within a valve.

3. The blocking component according to claim 2, wherein the fastener is selected from the group consisting of: an adhesive, a set of one or more sutures, and a set of one or more clips.

4. The blocking component according to claim 2, wherein the fastener anchors the bulky blocking component body to a set of one or more native leaflets of the valve.

5. The blocking component according to claim 2, wherein the fastener is a clip that is secured onto the bulky blocking component body by a connective element selected from the group consisting of: sutures, adhesives, and staples; and
wherein when the clip anchors the bulky blocking component body, the clip clips on to a set of one or more native leaflets of the heart such that the bulky blocking component body anchors within the aperture of the valve.

6. The blocking component according to claim 5, wherein the clip includes an eye that can accommodate a wire therethrough, and wherein the clip can be opened by tautening the wire.

7. The blocking component according to claim 2, wherein the bulky blocking component body and fastener are incorporated into a delivery system.

8. The blocking component according to claim 7, wherein the delivery system is a transcatheter delivery system further comprising:
a catheter housing the bulky blocking component body and fastener within; and
a guide wire that passes through and extends beyond the catheter and provides a means to guide the catheter through a patient's cardiovascular system.

9. The blocking device according to claim 8, wherein the fastener is a clip that is secured onto the bulky blocking component body by a connective element selected from the group consisting of: sutures, adhesives, and staples;
wherein the clip includes an eye that can accommodate a wire therethrough; and
wherein the transcatheter delivery system further comprises an actuating wire that passes through the eye and is adapted to open the clip.

10. The blocking component according to claim 1, wherein the bulky blocking component is formed from a material selected from a group consisting of: pericardium, expanded polytetrafluoroethylene (ePTFE), polyethylene terephthalate (PET), nylon, and polymer foam.

11. A blocking component for the repair of valvular insufficiency, comprising:
a blocking component body adapted to situate within a gap of a regurgitant heart valve;
wherein the blocking component body has a pocket with upper perimeter dimensions such that when the blocking component body is situated within the aperture, the upper perimeter of the pocket fills the aperture and mitigates regurgitant flow; and
wherein the blocking component body is made of a flexible and conformable material such that when situated within the aperture, the blocking component body responds to the cycles of systole and diastole by expanding to fill the aperture when the local pressure at the valve is low, mitigating regurgitant flow, and contracting when the local pressure at the valve is high, allowing the forward flow of blood.

12. The blocking component according to claim 11 further comprising a fastener;
wherein, the fastener is adapted to anchor the blocking component body within the aperture by securing the blocking component body within a valve.

13. The blocking component according to claim 12, wherein the fastener anchors the blocking component body to a set of one or more native leaflets of the valve.

14. The blocking component according to claim 12, wherein the fastener is a clip that is secured onto the blocking component body by a connective element selected from the group consisting of: sutures, adhesives, and staples; and
wherein when the clip anchors the blocking component body, the clip clips onto one or more native leaflets of the heart such that the blocking component body anchors within the aperture of the valve.

15. The blocking component according to claim 14, wherein the clip includes an eye that can accommodate a wire therethrough, and wherein the clip can be opened by tautening the wire.

16. The blocking component according to claim 12, wherein the blocking component body and fastener are incorporated into a transcatheter delivery system comprising
a catheter housing the blocking component body and fastener within, and
a guide wire that passes through and extends beyond the catheter and provides a means to guide the catheter through a patient's cardiovascular system.

17. The blocking component according to claim 16, wherein the fastener is a clip and the clip includes an eye that can accommodate a wire therethrough; and
wherein the transcatheter delivery system further comprises an actuating wire that passes through the eye and is adapted to open the clip.

18. A method of mitigating valvular insufficiency, comprising:
situating a blocking component within a gap of a regurgitant heart valve such that the blocking component fills the aperture and mitigates regurgitant flow;
wherein the blocking component is made of a flexible and conformable material that responds to the cycles of systole and diastole by expanding to fill the aperture when the local pressure at the valve is low, mitigating regurgitant flow, and contracting when the local pressure at the valve is high, allowing the forward flow of blood; and
wherein the blocking component comprises a blocking component body.

19. The method according to claim 18, wherein the blocking component body has a pocket with upper perimeter dimensions configured to fill the aperture and mitigate regurgitant flow.

20. A method to repair valvular insufficiency, comprising:
approaching, via a patient's circulatory system, a regurgitant heart valve with a transcatheter delivery system;
wherein the transcatheter delivery system comprises:
a blocking component adapted to situate within a gap within the regurgitant valve and configured such that, when situated within the gap, the blocking component mitigates regurgitation across the valve;
a fastening clip secured to the blocking component, the fastening clip having an eye;
an actuating wire disposed through the eye; and
a catheter defining an internal volume into which the blocking component, the fastening clip, and the actuating wire are disposed;
advancing the blocking device and the clip with actuating wire out of the catheter and towards a native leaflet proximate to the gap;
opening the clip by tautening the actuating wire; and
clipping the clip onto the native leaflet such that the blocking component is disposed in a configuration to fill the aperture and mitigate regurgitant flow.
